(19) United States
(12) Patent Application Publication
(10) Pub. No.: US 2025/0035518 A1
(43) Pub. Date: Jan. 30, 2025

(54) SAMPLE PROCESSING APPARATUS, SYSTEMS, AND METHODS

(71) Applicant: Porex Corporation, Fairburn, GA (US)

(72) Inventors: Avi Robbins, Atlanta, GA (US); Christopher John Ruf, Sandy Springs, GA (US); Brian VanHiel, Smyrna, GA (US); Maureen Carroll, Atlanta, GA (US)

(21) Appl. No.: 18/758,557

(22) Filed: Jun. 28, 2024

Related U.S. Application Data
(60) Provisional application No. 63/516,436, filed on Jul. 28, 2023.

(51) Int. Cl. G01N 1/10; G01N 1/28

(52) U.S. Cl. CPC G01N 1/10; G01N 1/28

(57) ABSTRACT

A liquid sample processing method includes opening a closed container by removing a closure of the container from an opening of the container to access a liquid sample collector including a liquid sample collection member and an elongated handle. Before removing the closure, the container may be secured with a holder. After separating the liquid sample collection member, a plunger with a liquid pathway extending through it is pushed downward to compress the liquid sample collection member such that at least some of the liquid sample flows through the liquid pathway.

### CROSS-REFERENCE

This patent application claims priority to and the benefit of the filing date of U.S. provisional patent application Ser. No. 63/516,436 filed Jul. 28, 2023 for “Sample Processing Apparatus, Systems, and Methods,” the entire contents of which is incorporated herein by reference.

### BACKGROUND

Some prior art sample collection techniques use a sample collector that is transported in a tube or other container. The collector may include a handle and a collection pad (e.g. for collecting a saliva sample by inserting the collection pad into the donor's mouth until a sufficient amount of saliva is absorbed by the pad). After collection the sample collector is placed in the transport tube, which may include a buffer or other liquid for preserving the collected sample. The tube is capped to seal it in a fluid and airtight manner. The capped tube is subsequently sent to a lab or other facility for processing. Processing typically involves: (1) de-capping, (2) separating the collection pad from the handle, (3) compressing the collection pad to expel and optionally filter the sample in the tube, and (4) recapping the tube for subsequent processing. Typically, all of these steps are done manually in a manner that leaves room for improvement.

### SUMMARY

We have developed sample processing apparatus, systems, and methods that improve on the prior art, and, in at least some instances, provide for processing that is faster, less strenuous, more efficient, and less risk of spillage or contamination that prior art processes.

In one example a liquid sample processing method includes receiving a closed container, the closed container containing a liquid sample collector having a liquid sample collection member connected to an elongated handle. The close container is opened by removing a closure of the container from an opening of the container. After opening the closed container, the liquid sample collection member is separated from the elongated handle. A plunger body is inserted into the container above the separated liquid sample collection member, the plunger body having a liquid pathway extending through the plunger body. The plunger body is pushed downward to compress the liquid sample collection member such that the liquid sample collection member expels at least some of the liquid sample. The liquid sample is expelled such that at least some of the liquid sample flows through the liquid pathway.

In some implementations the process includes positioning the separated elongated handle between the plunger body and the closure of the container and then replacing the closure into the opening of the container.

In some implementations an end of the elongated handle is secured to the closure as the plunger body is pushed downward.

In some implementations replacing the closure causes a second end of the elongated handle to contact the plunger body to push the plunger body downward.

In some implementations, when the second end of the elongated handle contacts the plunger body to push the plunger body downward, the second end of the elongated handle spans across the liquid pathway of the plunger body.

In some implementations, after pushing the plunger body downward, the closure has been replaced, the elongated handle of the liquid sample collector extends from the closure to the plunger body, and the plunger body retains the liquid sample collection member in a compressed state at a bottom of the container.

In some implementations the liquid sample is a saliva sample.

In some implementations the liquid sample collection member is a collection pad mounted at an end of the elongated handle.

In some implementations opening the closed container includes prying the closure from the opening using a prying tool inserted into a cavity extending into the closure.

In some implementations opening the closed container includes, prior to prying the closure from the opening, securing the closed container with a holder.

In some implementations the holder includes a base secured to a surface, wherein the holder further comprises at least two spaced apart holding members extending upwardly from the base, the holding members sized and shaped to hold a bottom of the container.

In some implementations the liquid sample collection member is separated from the elongated handle while the liquid sample collection member remains in the container.

In some implementations the liquid sample collection member is separated from the elongated handle using a separation tool, the separation tool including an elongated handle and teeth at an end of the elongated handle configured to engage the liquid sample collection member.

In some implementations the plunger body includes a filter in the liquid pathway.

In some implementations the liquid sample is expelled such that at least some of the liquid sample flows through the filter in the liquid pathway.

In some implementations the plunger body is a cylinder with a hollow center, the hollow center defining the liquid pathway.

In some implementations the plunger body is sized and shaped for a friction fit with an interior of the container.

In some implementations the plunger body includes protrusions positioned for a friction fit with the interior of the container.

In some implementations the liquid sample is expelled such that at least some of the liquid sample is above the plunger body after pushing the plunger body downward.

### BRIEF DESCRIPTION OF FIGURES

FIG. 1 shows an example of a container for a liquid sample.

FIGS. 2-4 show an example of opening a container for a liquid sample.

FIGS. 5-7 show an example of separating a liquid sample collection member from an elongated handle.

FIG. 8 shows an example of inserting a plunger body into the container.

FIGS. 9-10 show an example of pushing the plunger body downward to compress the liquid sample collection member.

FIG. 11 shows the container for a liquid sample of FIG. 1 after compression of a liquid sample collection member.

FIGS. 12-14 show an example of using a separation tool to separate an elongated handle from a closure.

### DETAILED DESCRIPTION

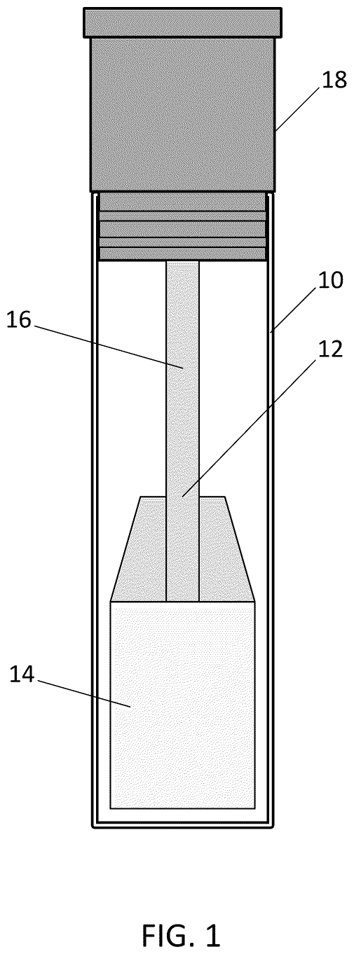

FIG. 1 shows an example of a closed container 10 for a liquid sample. The closed container 10 contains a liquid sample collector 12 that has been used to collect a liquid sample. The liquid sample collector 12 includes a liquid sample collection member 14 connected to an elongated handle 16. The container 10 is closed by a removable closure 18. In some implementations the container 10 may be a vial and the closure 18 may be a cap that removably secures to the vial in a fluid tight fashion.

The liquid sample collection member 14 may be configured to collect a liquid sample. In some implementations the liquid sample collection member 14 may be a pad formed of a material and otherwise configured to absorb a saliva sample (e.g. such as by inserting the liquid sample collection member 14 into a sample donor's mouth). In some implementations the liquid sample collection member 14 may be a pad formed of a material and otherwise configured such that compression of the liquid sample collection member 14 causes it to release the absorbed liquid sample.

In other implementations the liquid that may be absorbed by the liquid sample collection member 14 may be any liquid to be collected and/or tested. Non-limiting examples include but are not limited to oral fluid, saliva, urine, blood, diarrhea/feces, sweat, vaginal fluids, semen, or any other appropriate fluid. Although described in connection with healthcare, it should be understood that the disclosed device may be used for a myriad of other fluid collection uses. The liquid sample collection member 14 may be associated with a sufficiency indicator that can change color or otherwise provide a visual indication that the liquid sample collection member 14 is sufficiently impregnated with the liquid to be collected.

The liquid sample collection member 14 is intended to absorb and hold liquid, but to also release the held liquid upon compression. The material of the liquid sample collection member 14 may be formed of fibers, foams, or particulate materials made from polyesters, polyurethanes, polyethylene, polypropylene, PTFE, PVDF, elastomeric materials, thermoplastic polyurethane, thermoplastic polyethylene, nylon, cellulose, cellulose acetate, natural fibers, fabric, paper, polymer hydrogel (dry form and/or wet form) such as HEMA, NIPAM, etc., a natural or man-made material, sponge, cotton, or any combination thereof. The liquid sample collection member 14 may be treated with dried buffer, saliva-production stimulants such as citrates, materials that prevent target analyte binding to the material, surfactants to help collect and release liquid, or any other appropriate material, or any combination thereof.

As shown in FIG. 1, the liquid sample collector 12 may be connected to the closure 18 of container 10 at an end of the elongated handle 16, such that the liquid sample collector 12 is suspended inside of the container 10 when it is in a closed state.

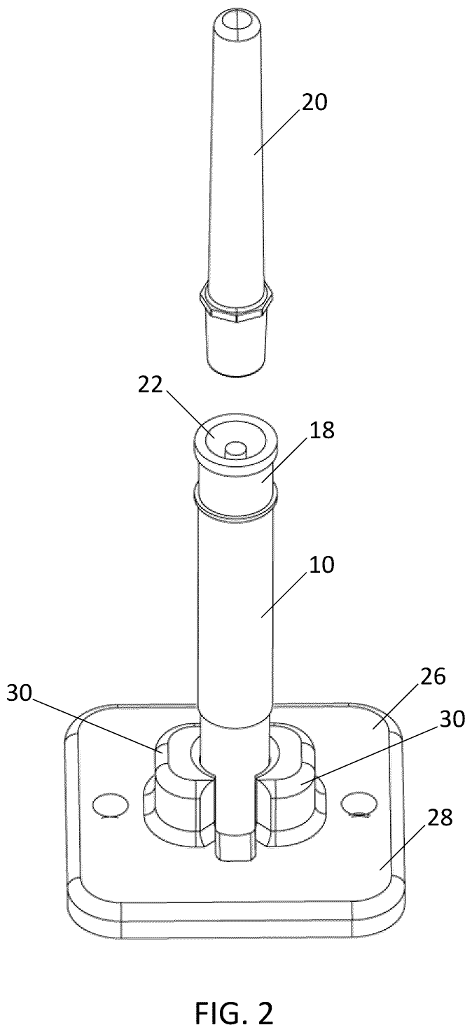

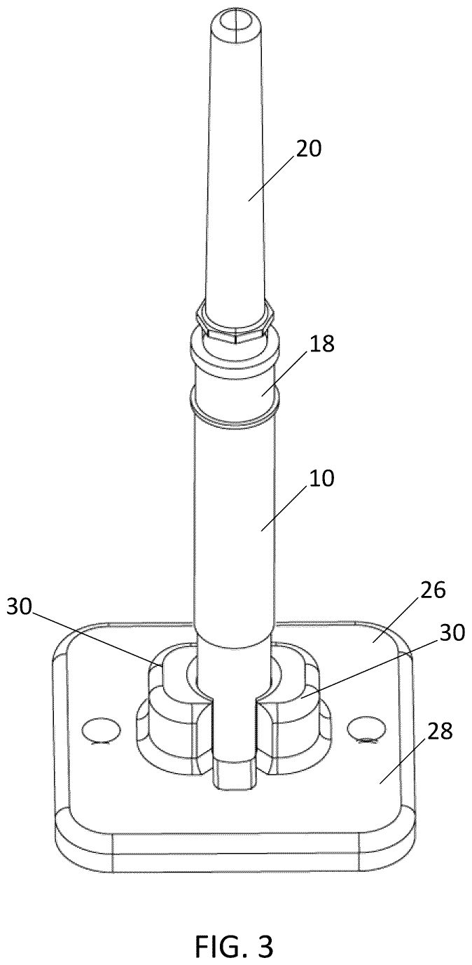

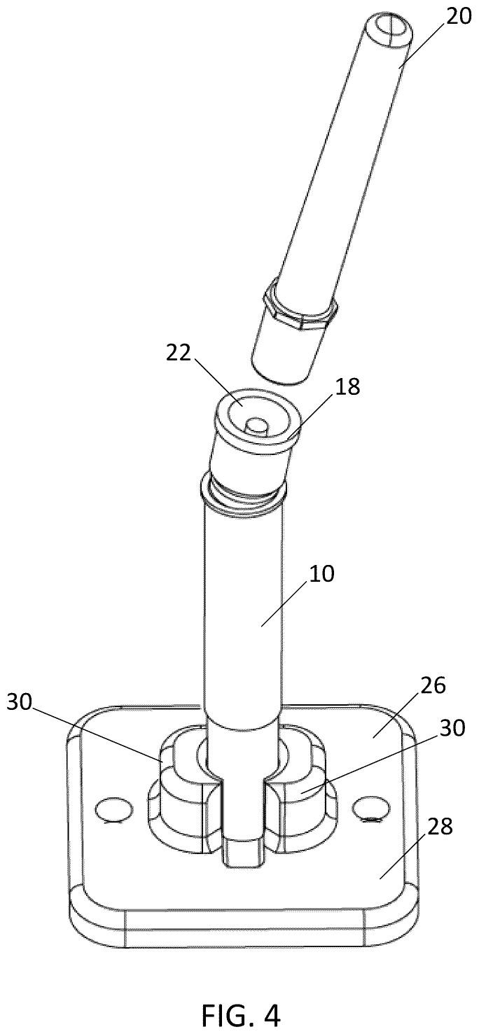

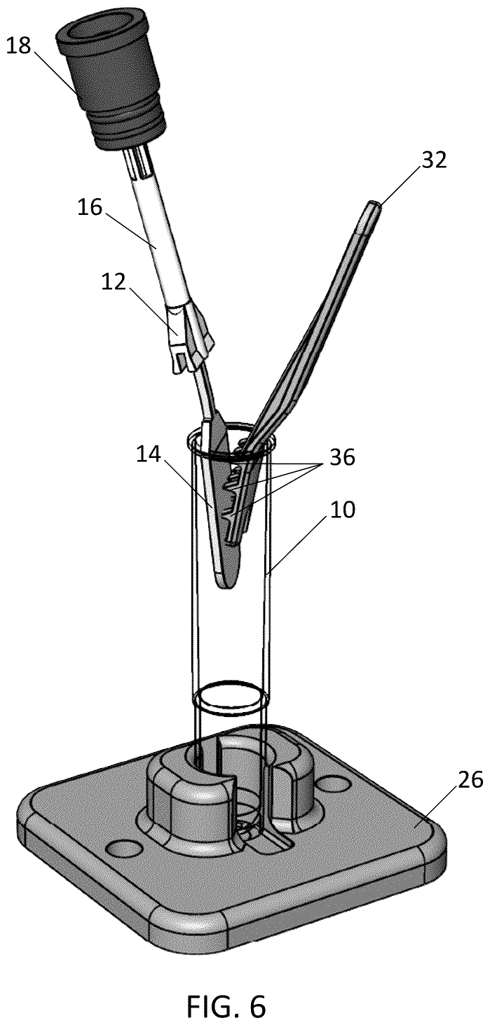

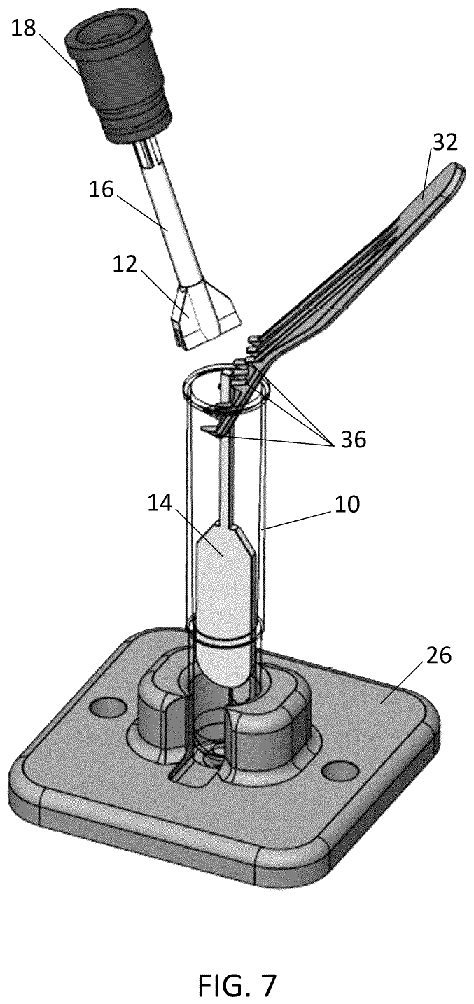

FIGS. 2-10 show an example of a liquid sample processing method for a liquid sample held in the container of FIG. 1.

FIGS. 2-4 show an example of opening the liquid sample container 10 by removing the closure 18 from an opening of the container 10. In this example, a prying tool 20 is used to pry the closure 18 from the opening of the container 10. The prying tool 20 may be used to open the container 10 in a controlled manner, reducing risk that liquid sample may be unintentionally expelled from the container 10 during opening by hand, or that the liquid sample collector 12 may be unintentionally disconnected from the closure 18 during opening by hand.

The prying tool 20 is inserted into a cavity 22 extending into the closure 18 and is then tilted to pry the closure 18 from the container 10. In the particular example shown the prying tool 20 has an end 24 that is shaped to closely match the cavity 22 in the closure 18 such that tilting the prying tool 20 relative to the container 10 will force the closure 18 to also tilt relative to the container 10 thereby releasing the closure 18 from the container 10 in a controlled fashion. In other implementations, the end 24 of prying tool 20 and/or the cavity 22 of the closure 18 may be shaped and otherwise configured in other fashions while still allowing for interaction of the prying tool 20 with the closure 18 such that tilting the prying tool 20 relative to the container 10 will force the closure 18 to also tilt relative to the container 10 thereby releasing the closure 18 from the container 10. After releasing the closure 18 from the container 10, the prying tool 20 may be twisted relative to the closure 18 to release it from the closure 18.

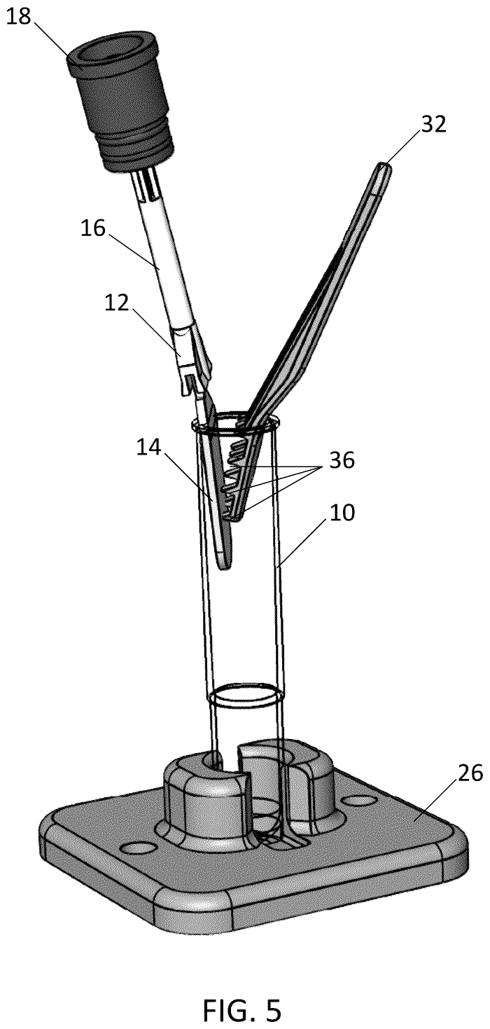

As also shown in FIGS. 2-4, prior to prying the closure 18 from the opening of container 10, the container 10 may be secured using a holder 26. In the particular example shown, the holder 26 may include a base 28 configured to be secured to a surface (e.g. a top of a table) and two spaced apart holding members 30 extending upwardly from the base 28. The holding members 30 are sized and shaped to hold a bottom of the container 10. In some implementations the holder 26 supports the container 10 in an upright orientation and stabilizes the container 10 while the prying tool 20 is used to pry the closure 18 from the container 10. As shown in FIGS. 5-10, the container 10 may remain secured in the holder 26 for subsequent steps of the process.

After removing the closure 18, the liquid sample collection member 14 may be separated from the elongated handle 16 of the liquid sample collector 12. The liquid sample collection member 14 may be separated from the elongated handle 16 while the elongated handle 16 remains connected to the closure 18.

FIGS. 5-7 show an example of separating the liquid sample collection member 14 from the elongated handle 16. In this example the liquid sample collection member 14 (which is a collection pad mounted at the end of the elongated handle 16) is separated from the elongated handle 16 while the liquid sample collection member 14 remains inside the container 10. The liquid sample collection member 14 is separated from the elongated handle 16 using a separation tool 32. The separation tool 32 in the example of FIGS. 5-7 includes an elongated handle 34 and several teeth 36 at the end of the elongated handle 34. The teeth 36 are configured to engage the liquid sample collection member 14, allowing it to be pulled from the elongated handle 16 of the liquid sample collector 12 such that the liquid sample collection member 14 remains in the container 10 as the elongated handle 16 of the liquid sample collector 12 is pulled away from the container 10.

In some implementations the teeth 36 of the separation tool 32 may be engaged into the liquid sample collection member 14 as the closure 18 and connected elongated handle 16 of the liquid sample collector 12 are pulled upwardly, thereby separating the liquid sample collection member 14 from the elongated handle 16. Subsequently, the liquid sample collection member 14 may be allowed to fall to the bottom of the container 10.

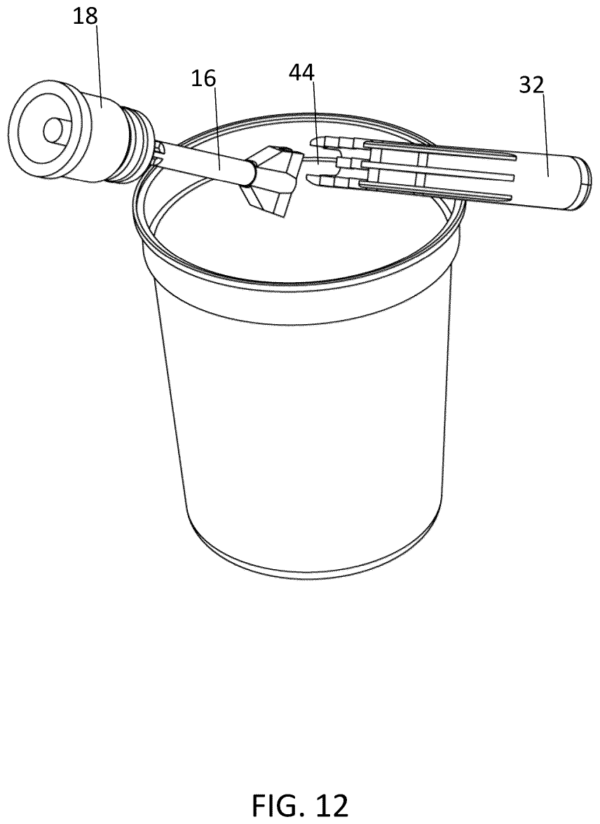

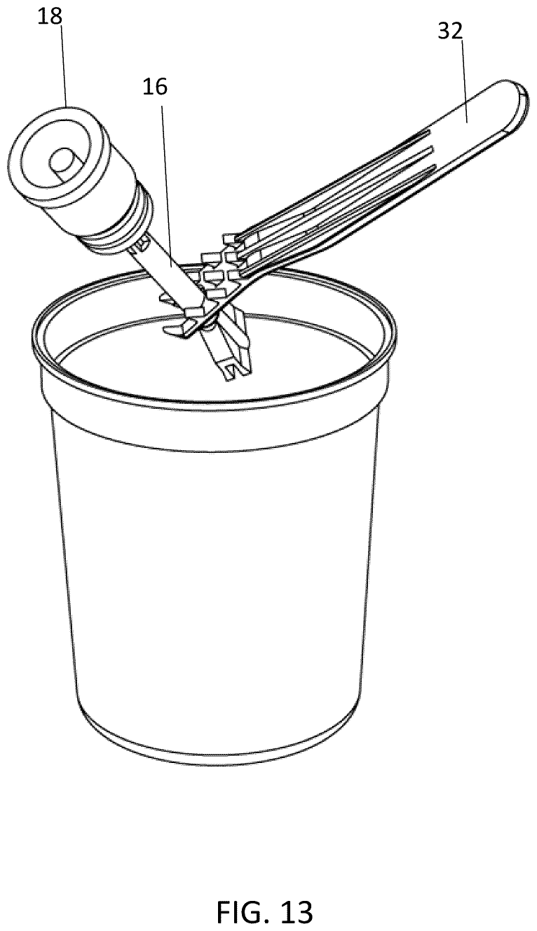

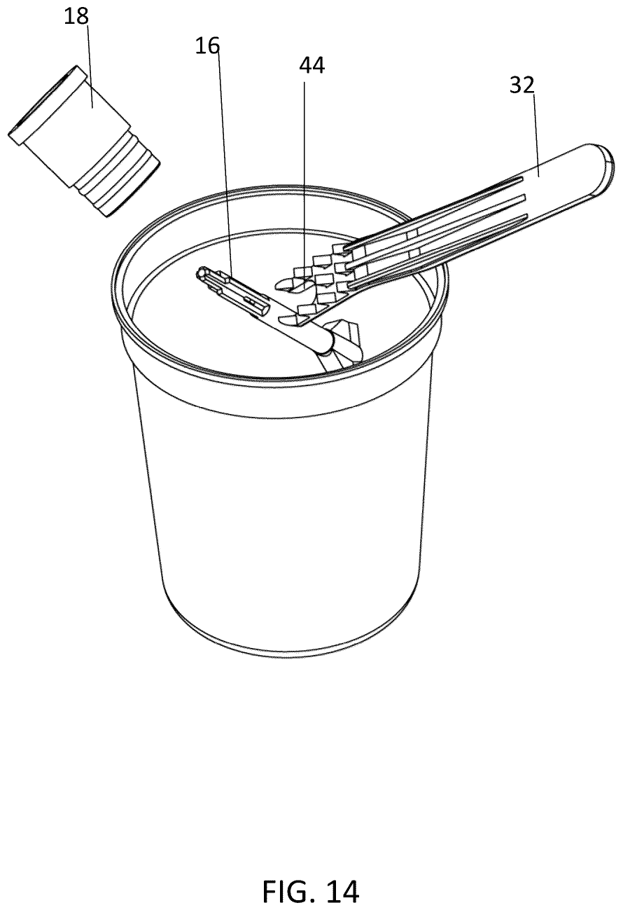

In some implementations the separation tool 32 may also be used to separate the elongated handle 16 from the closure 18 as shown in FIGS. 12-14. In such implementations a slot 44 in the separation tool 32 may be used to engage the elongated handle 16 and pull the elongated handle 16 from the closure 18 without the user having to touch the elongated handle 16.

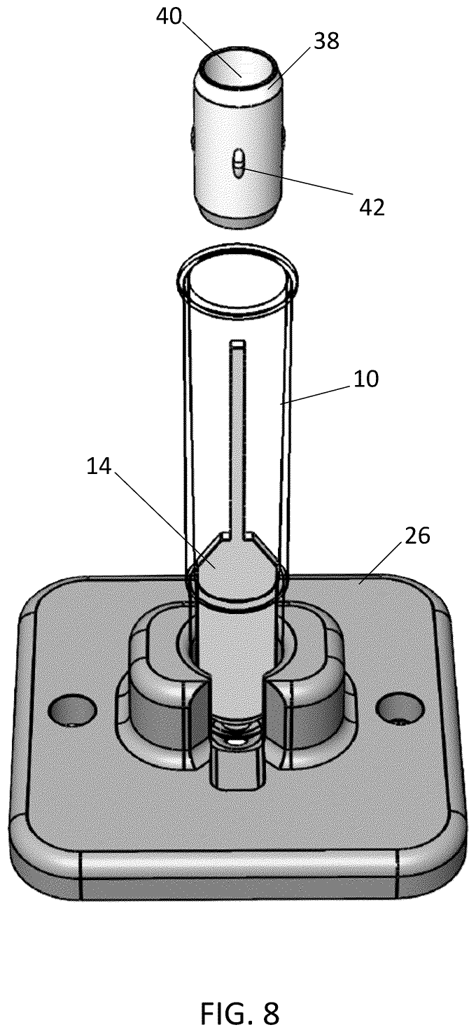

After the liquid sample collection member 14 is separated from the elongated handle 16 of the liquid sample collector 12, with the liquid sample collection member 14 remaining in the container 10, a plunger body 38 may be inserted into the container 10 above the liquid sample collection member 14. One example of this is shown in FIG. 8. The plunger body 38 shown in FIG. 8 includes a liquid pathway 40 extending through the plunger body 38 from a distal end to a proximal end. In some implementations the liquid pathway 40 may include a filter (e.g. a porous media). In other implementations the liquid pathway 40 may be hollow. In the particular example shown in FIG. 8 the plunger body 38 is a cylinder with a hollow center, with the hollow center defining the liquid pathway 40. In the particular example shown in FIG. 8, the plunger body 38 is sized and shaped for the friction fit with an interior of the container 10. More specifically, the plunger body 38 in this example includes protrusions 42 that interact with the interior of the container to provide a friction fit (albeit a slidable friction fit) with the interior of the container 10.

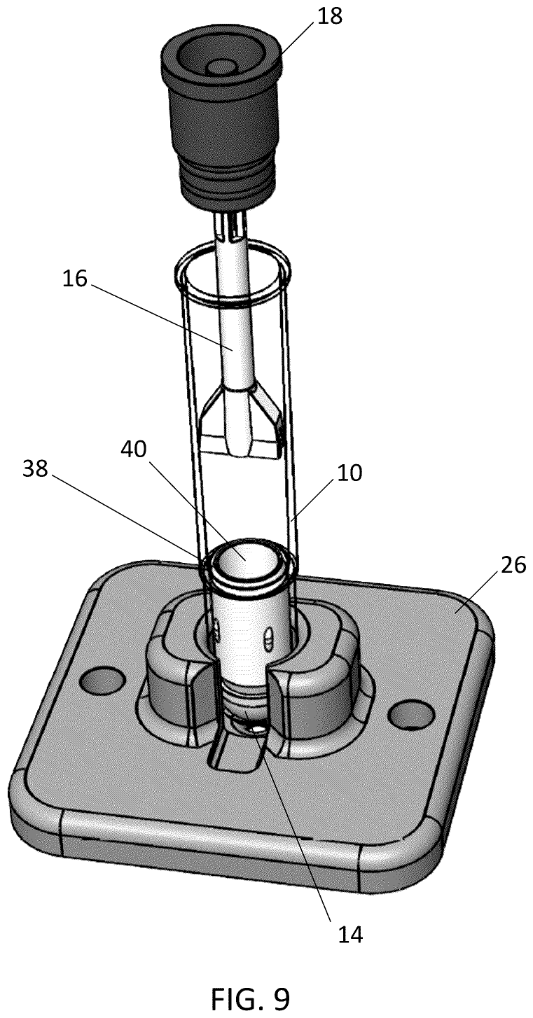

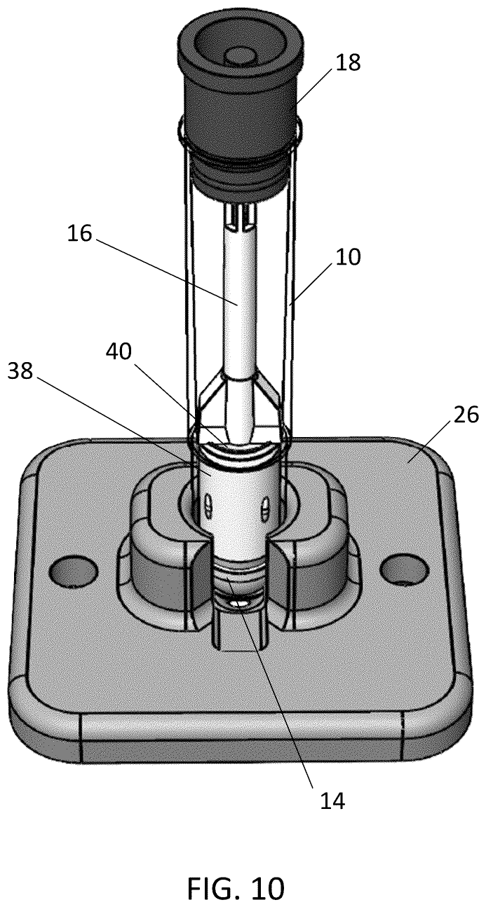

After the plunger body 38 is inserted into the container 10 above the separated liquid sample collection member 14, the plunger body 38 may be pushed downward to compress the liquid sample collection member 14 such that the liquid sample collection member 14 expels at least some of its liquid sample. Furthermore, the liquid sample may be expelled such that at least some of the liquid sample flows through the liquid pathway 40 of the plunger body 38. FIGS. 9 and 10 show one example of this.

In FIGS. 9 and 10, the separated elongated handle 16 of the liquid sample collector 12 is positioned between the plunger body 38 and the closure 18 of the container 10 and then the closure 18 is replaced into the opening of the container 10. In some implementations, the elongated handle 16 remains attached to the closure 18 through the entirety of the process, as shown in FIGS. 2-10, and thus “positioning” the elongated handle 16 between the plunger body 38 and closure 18 constitutes manipulating the closure 18 to reinsert the elongated handle 16 into the container 10. In FIGS. 9 and 10, after the elongated handle 16 is positioned between the plunger body 38 and the closure 18, the closure 18 is replaced into the opening of the container 10.

In this example, replacing the closure 18 with the elongated handle 16 attached allows the elongated handle 16 to contact the plunger body 38 to push the plunger body 38 downward. The end of the elongated handle 16 that contacts the plunger body 38 is widened, such that it spans across the liquid pathway 40 of the plunger body 38.

As shown in FIGS. 9 and 10, the closure 18, the elongated handle 16, the plunger body 38, the container 10, and the liquid sample collection member 14 are all sized and otherwise configured such that replacing the closure 18 with the elongated handle 16 attached causes the plunger body 38 to be pushed downward thereby compressing the liquid sample collection member 14. In this example, when the closure 18 is replaced, the elongated handle 16 spans between where it is connected at one end to the closure 18 and its other end, where it holds the plunger body towards the bottom of the container 10 such that the liquid sample collection member 14 is maintained in a compressed state.

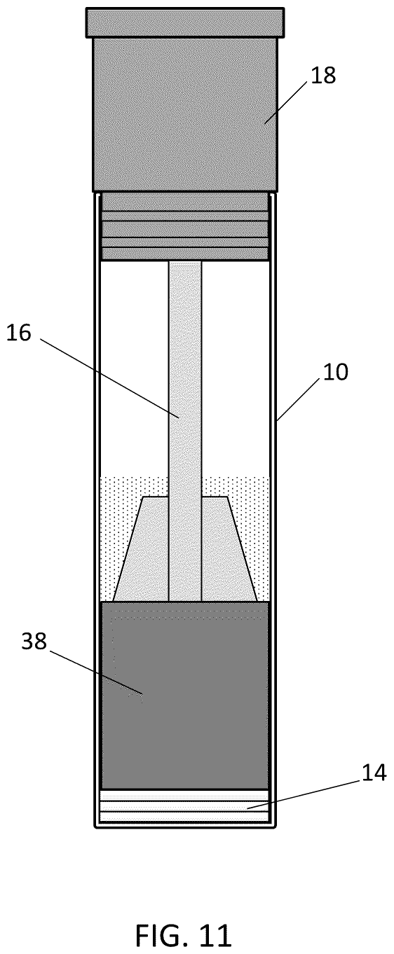

When the liquid sample collection member 14 is compressed, it will expel at least some of the liquid sample such that at least some of the liquid sample will flow upwards through the liquid pathway 40 in the plunger body 38. In implementations in which the liquid pathway 40 includes a filter, at least some of the expelled liquid sample may flow through the filter as a filtrate. After compression of the liquid sample collection member 14, at least some of the expelled liquid sample may be located above the plunger body 38, as shown in FIG. 11.

The above description is provided by way of example only. Additions, deletions, substitutions, and other changes or modifications may be made to the above-described examples without departing from the scope or spirit of the inventions set out in the following claims.

1. A liquid sample processing method comprising:
(a) receiving a closed container, the closed container containing a liquid sample collector comprising a liquid sample collection member connected to an elongated handle;
(b) opening the closed container by removing a closure of the container from an opening of the container;
(c) after opening the closed container, separating the liquid sample collection member from the elongated handle;
(d) inserting a plunger body into the container above the separated liquid sample collection member, wherein the plunger body comprises a liquid pathway extending through the plunger body; and
(e) pushing the plunger body downward to compress the liquid sample collection member such that the liquid sample collection member expels at least some of the liquid sample, wherein the liquid sample is expelled such that at least some of the liquid sample flows through the liquid pathway.

2. The liquid sample processing method of claim 1 wherein pushing the plunger body downward comprises positioning the separated elongated handle between the plunger body and the closure of the container and then replacing the closure into the opening of the container.

3. The liquid sample processing method of claim 2 wherein an end of the elongated handle is secured to the closure as the plunger body is pushed downward.

4. The liquid sample processing method of claim 3 wherein replacing the closure causes a second end of the elongated handle to contact the plunger body to push the plunger body downward.

5. The liquid sample processing method of claim 4 wherein, when the second end of the elongated handle contacts the plunger body to push the plunger body downward, the second end of the elongated handle spans across the liquid pathway of the plunger body.

6. The liquid sample processing method of claim 1 wherein, after pushing the plunger body downward, the closure has been replaced, the elongated handle of the liquid sample collector extends from the closure to the plunger body, and the plunger body retains the liquid sample collection member in a compressed state at a bottom of the container.

7. The liquid sample processing method of claim 1 wherein the liquid sample comprises a saliva sample.

8. The liquid sample processing method of claim 1 wherein the liquid sample collection member comprises a collection pad mounted at an end of the elongated handle.

9. The liquid sample processing method of claim 1 wherein opening the closed container comprises prying the closure from the opening using a prying tool inserted into a cavity extending into the closure.

10. The liquid sample processing method of claim 9 wherein opening the closed container comprises, prior to prying the closure from the opening, securing the closed container with a holder.

11. The liquid sample processing method of claim 10 wherein the holder comprises a base on a surface, wherein the holder further comprises at least two spaced apart holding members extending upwardly from the base, the holding members sized and shaped to hold a bottom of the container.

12. The liquid sample processing method of claim 1 wherein the liquid sample collection member is separated from the elongated handle while the liquid sample collection member remains in the container.

13. The liquid sample processing method of claim 12 wherein the liquid sample collection member is separated from the elongated handle using a separation tool, the separation tool comprising an elongated handle and a plurality of teeth at an end of the elongated handle configured to engage the liquid sample collection member.

14. The liquid sample processing method of claim 1, wherein the plunger body further comprises a filter in the liquid pathway.

15. The liquid sample processing method of claim 14 wherein the liquid sample is expelled such that at least some of the liquid sample flows through the filter in the liquid pathway.

16. The liquid sample processing method of claim 1 wherein the plunger body comprises a cylinder with a hollow center, the hollow center defining the liquid pathway.

17. The liquid sample processing method of claim 1 wherein the plunger body is sized and shaped for a friction fit with an interior of the container.

18. The liquid sample processing method of claim 17 wherein the plunger body comprises a plurality of protrusions positioned for a friction fit with the interior of the container.

19. The liquid sample processing method of claim 1 wherein the liquid sample is expelled such that at least some of the liquid sample is above the plunger body after pushing the plunger body downward.
